(19) United States
(12) Patent Application Publication
(10) Pub. No.: US 2014/0296688 A1
(43) Pub. Date: Oct. 2, 2014

(54) SURFACE DEFORMATION SENSOR

(71) Applicants: David Chuen Chun Lam, Hong Kong (CN); Guozhen Chen, Hong Kong (CN); Ion Seng Chan, Hong Kong (CN)

(72) Inventors: David Chuen Chun Lam, Hong Kong (CN); Guozhen Chen, Hong Kong (CN); Ion Seng Chan, Hong Kong (CN)

(73) Assignee: THE HONG KONG UNIVERSITY OF SCIENCE AND TECHNOLOGY, Hong Kong (CN)

(21) Appl. No.: 14/117,623

(22) PCT Filed: May 15, 2012
(86) PCT No.: PCT/CN12/00653

§ 371 Date: Nov. 13, 2013

Related U.S. Application Data
(60) Provisional application No. 61/457,796, filed on Jun. 6, 2011.

(51) Int. Cl. A61B 3/16; A61B 3/107

(52) U.S. Cl. CPC A61B 3/16; A61B 3/107
USPC 600/399; 600/405

(57) ABSTRACT

A surface deformation sensor that includes a resonance circuit is described herein. The resonance circuit includes a sensing capacitor and inductive coil. The resonance circuit receives an external signal, which causes the resonance circuit to resonate at a resonance frequency. A surface deformation of an object can be determined based on the resonance frequency.

### CROSS REFERENCE TO RELATED APPLICATIONS

This application claims priority to U.S. provisional application No. 61/457,796, entitled: “CAPACITANCE-BASED LOW FORCE CURVATURE SENSOR WITH APPLICATION IN NON-INVASIVE CORNEAL MONITORING” and filed on Jun. 6, 2011.

### TECHNICAL FIELD

This disclosure generally relates to measurement of surface deformation utilizing a capacitance-based sensor.

### BACKGROUND

The World Health Organization ranks glaucoma as the second leading cause of blindness in the world. In the United States, glaucoma accounts for 9-12% of all cases of blindness. Glaucoma patients have peaks and fluctuations of intraocular pressure, which are associated with progression of vision loss.

Currently, ophthalmologists and optometrists do not test a person's intraocular pressure frequently. Intraocular pressure varies according to a circadian (24-hour) cycle. A person may exhibit normal intraocular pressure upon examination, but exhibit fluctuations to abnormal intraocular pressure at other times. This causes many people to go undiagnosed with early stages of glaucoma. Infrequent monitoring also leads to difficulty monitoring the course of glaucoma.

The above-described background is merely intended to provide an overview of contextual information regarding glaucoma, and is not intended to be exhaustive. Additional context may become apparent upon review of one or more of the various non-limiting embodiments of the following detailed description.

### SUMMARY

The following presents a simplified summary of the specification in order to provide a basic understanding of some aspects of the specification. This summary is not an extensive overview of the specification. It is intended to neither identify key or critical elements of the specification nor delineate any scope of particular embodiments of the specification, or any scope of the claims. Its sole purpose is to present some concepts of the specification in a simplified form as a prelude to the more detailed description that is presented later.

In accordance with one or more embodiments and corresponding disclosure, various non-limiting aspects are described in connection with a capacitance-based sensor used to measure surface deformation. The sensor described herein can be utilized to achieve continuous intraocular pressure monitoring by measuring curvature deformation of the cornea in a non-invasive manner.

In accordance with a non-limiting embodiment, a sensor is described that can be used to measure surface deformation. The sensor includes a reference layer and a deformation layer. The reference layer includes an upper electrode and an inductive coil. The deformation layer includes a lower electrode. The lower electrode and the upper electrode form a sensing capacitor. The inductive coil is electrically coupled to the sensing capacitor and produces a resonance in response to an external electromagnetic force.

In another non-limiting embodiment, a bio-compatible sensor is described that can be used to measure surface deformation in eye tissue. For example, the bio-compatible sensor can be used to determine fluctuations in intraocular pressure based on surface deformation in cornea tissue. The bio-compatible sensor includes a capacitor and an inductive coil. The capacitor includes a rigid layer with an upper electrode and a soft deformable layer with a lower electrode. The inductive coil is formed on the rigid layer and is electrically coupled to the capacitor to form a resonant circuit. The resonant circuit resonates at a resonance frequency that is proportional to the capacitance and is measurable when the resonant circuit is excited by an electromagnetic signal.

In a further non-limiting embodiment, a method is described for measuring surface deformation. An external signal can be received at a resonance circuit. The resonance circuit can include a sensing capacitor and an inductive coil. The sensor is energized by the external signal so that the resonance circuit resonates at a resonance frequency based on the external signal. A surface deformation of an object based on the resonance frequency.

In another non-limiting embodiment, a system is described for measuring surface deformation. The system includes means for energizing a sensing capacitor so that a resonance circuit resonates at a resonance frequency based on an external signal. The system also includes means for determining a surface deformation of an object based on the resonance frequency.

The following description and the drawings set forth certain illustrative aspects of the specification. These aspects are indicative, however, of but a few of the various ways in which the various embodiments of the specification may be employed. Other aspects of the specification will become apparent from the following detailed description of the specification when considered in conjunction with the drawings.

### BRIEF DESCRIPTION OF THE DRAWINGS

Numerous aspects and embodiments are set forth in the following detailed description, taken in conjunction with the accompanying drawings, in which like reference characters refer to like parts throughout, and in which:

FIG. 1 illustrates an example non-limiting cross-sectional view of a surface deformation sensor, according to an embodiment of the disclosure;

FIG. 2 illustrates an example non-limiting top view of the surface deformation sensor, according to an embodiment of the disclosure;

FIG. 3 illustrates an example non-limiting top view of the rigid layer of the surface deformation sensor, according to an embodiment of the disclosure;

FIG. 4 illustrates an example non-limiting top view of the soft layer of the surface deformation sensor, according to an embodiment of the disclosure;

FIG. 5 illustrates an example non-limiting cross sectional view of the surface deformation sensor mounted on the surface of the eye, according to an embodiment of the disclosure;

FIG. 6 is an example non-limiting graph illustrating the relationship between deformations of the soft layer with layer thickness, according to an embodiment of the disclosure;

FIG. 7 is an example non-limiting graph illustrating the relationship between resonance frequency of a resonant circuit of the surface deformation sensor and intraocular pressure, according to an embodiment of the disclosure;

FIG. 8 is an is an example non-limiting graph illustrating measuring intraocular pressure with the surface deformation sensor and measuring intraocular pressure with a pressure sensor implanted into the eyeball, according to an embodiment of the disclosure;

FIG. 9 is an example non-limiting process flow diagram of a method for fabricating the rigid layer of the surface deformation sensor, according to an embodiment of the disclosure;

FIG. 10 is an example non-limiting process flow diagram of a method for determining a surface deformation of an object, according to an embodiment of the disclosure; and

FIG. 11 is an example non-limiting process flow diagram of a method for determining intraocular pressure, according to an embodiment of the disclosure.

### DETAILED DESCRIPTION

Various aspects or features of this disclosure are described with reference to the drawings, wherein like reference numerals are used to refer to like elements throughout. In this specification, numerous specific details are set forth in order to provide a thorough understanding of this disclosure. It should be understood, however, that the certain aspects of disclosure may be practiced without these specific details, or with other methods, components, materials, etc. In other instances, well-known structures and devices are shown in block diagram form to facilitate description and illustration of the various embodiments.

In accordance with one or more embodiments described in this disclosure, a sensor based on an inductor-capacitor resonance circuit is described that can detect surface deformation of an object. The sensor is a soft sensor structure that can be used to measure surface deformation of a soft object. The sensor can be applied to the soft object via low force application methods with minimal or no damage to the soft object. For example, the sensor can be utilized to measure surface deformation or curvature change in the cornea to achieve non-invasive continuous intraocular pressure monitoring.

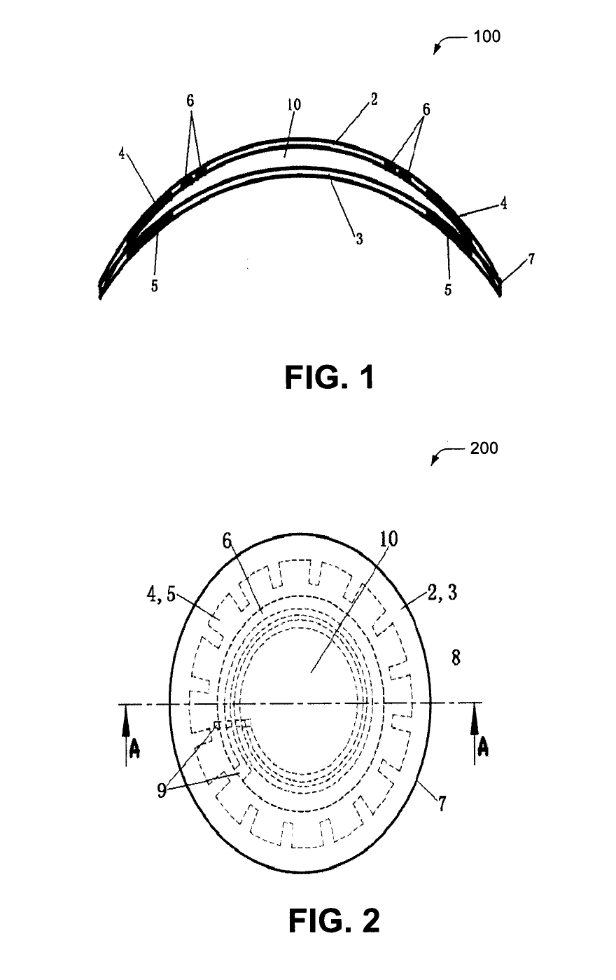

Referring now to the drawings, with reference initially to FIG. 1, a cross-sectional view 100 of a surface deformation sensor is illustrated, according to an embodiment of the disclosure. FIG. 2 illustrates a top view 200 (or plan view) of the surface deformation sensor, according to an embodiment of the disclosure. The surface deformation sensor is shown in FIG. 2 as being generally circular shaped. This is merely for simplicity of illustration. The surface deformation sensor can have any shape so that the surface deformation sensor is capable of being mounted to a surface of an object for measurement of surface deformation. In an embodiment, the sensor need only have a curvature substantially similar to that of the surface.

The surface deformation sensor includes a rigid layer 2 (upper layer, rigid layer, or reference layer) and a soft layer 3 (lower layer, deformation layer, or soft deformable layer). The rigid layer 2 can be made of a rigid or hard film. The soft layer 3 can be made of a soft deformable film. In an embodiment, the object can be a biological object (e.g., ocular tissue, such as the cornea) and the rigid layer 2 and the soft layer 3 are made, at least partially, of a bio-compatible material. A bio-compatible material is a material that has been approved by the Food and Drug Administration of the United States as safe for prolonged human contact.

The rigid layer 2 and the soft layer 3 can be bounded together at the edge 7. Although edge 7 is illustrated at the right edge, it will be understood that this is merely for simplicity of illustration; edge 7 can be any portion of external edge of the sensor. For example, in FIG. 2, edge 7 can include at least a portion of the external circumference of the sensor.

The soft layer 3 can contact the surface of an object. The soft layer 3 can change shape in response to surface deformation of the object. According to an embodiment, the sort layer 3 can change shape conformally with deformation of the surface of the object. The sensor includes a gap 10 between the rigid layer 2 and the soft layer 3. The gap 10 is capable of changing as the shape of the soft layer 3 change shape in response to surface deformation of the object. In an embodiment, the size of the gap varies with a change in topology of the object (surface deformation of the object). The rigid layer 2 does not substantially change shape in response to the surface deformation of the object.

According to an embodiment, gap 10 can be at least partially filed with a dielectric material. The dielectric material can be any deformable dielectric material. For example, the dielectric material can be a gel, a fluid, a gas, or the like. When the object is a biological object, the dielectric material can be a bio-compatible gel, a bio-compatible fluid, a bio-compatible gas, or any other bio-compatible material.

The rigid layer 2 and the soft layer 3 can have a curvature similar to that of the object. Having similar curvature can facilitate the sensor being easily mounted onto the surface of the object with minimal external force. In an embodiment, the sensor can be mounted onto the surface of the object without external force.

The rigid layer 2 is made of one or more rigid materials. According to an embodiment, the rigid layer 2 is made of a rigid silicon material. The rigid layer 2 includes an upper electrode 4 and an inductive coil 6. The upper electrode 4 and/or the inductive coil 6 can be fabricated inside the rigid layer 2. The upper electrode 4 and/or the inductive coil 6 can also be placed on the surface of the rigid layer 2. In an embodiment, the upper electrode 4 is a conductive thin film. The inductive coil 6 can be a conductive wire or a semiconductor wire.

The soft layer 3 is made of one or more soft materials. According to an embodiment, the soft layer 3 is made of a soft silicon rubber. The soft layer 3 includes a lower electrode 5. The lower electrode 5 can be fabricated inside the soft layer 3. The lower electrode 5 can also be placed on the surface of the soft layer 3. In an embodiment, the lower electrode 5 can be a conductive thin film.

The soft layer 3 can also include a mechanism 9 to electrically connect or couple the inductive coil 6 and the lower electrode 5. The mechanism 9 can be a wire, such as a short bounding wire. It will be understood that the mechanism 9 can be any mechanism that facilitates an electrical connection between the inductive coil 6 and the lower electrode 5. The mechanism 9 can be fabricated inside the soft layer 3. The mechanism 9 can also be fabricated on the surface of the soft layer 3.

The lower electrode 5 can form a sensing capacitor with the upper electrode 4. The inductive coil 6 can be electrically coupled to the sensing capacitor through the mechanism 9. The inductive coil 6 and the sensing capacitor can form a resonant circuit (LC tank circuit). The resonant circuit can resonate in response to exposure to an external electromagnetic field. In an example, the inductive coil 6 can resonate in response to the external electromagnetic field as part of the resonant circuit.

The soft layer 3 can change shape according to a surface deformation of an object. The shape change of the soft layer 3 causes the distance between the upper electrode 4 and the lower electrode 5 to change. The change in distance causes the capacitance to change. The change in capacitance causes the inductive coil 6 to resonate at a resonance frequency. The resonance frequency depends on the change in capacitance, which depends on the shape change of the soft layer 3, which corresponds to the surface deformation. In other words, the resonance varies proportionally with a change in the topology of the object (surface deformation).

In an embodiment, the resonant circuit (LC tank circuit) including the inductive coil 6 and the capacitive element formed by the upper electrode 4 and the lower electrode 5 can be excited in response to an external electromagnetic signal in the radio frequency (RF) range. Resonant circuits of this type have a natural resonant frequency fₒ, that, to the first order, depends on the value of the inductive coil 6 and the capacitor as:

f₀=1/2π\sqrt{LC}

where L is the inductance of the inductive coil 6 and C is the capacitance of the capacitive element formed by the upper electrode 4 and the lower electrode 5. Accordingly, as the capacitance of the curvature sensor changes, the resonance frequency fₒ of the resonance circuit will change proportionally to the surface deformation.

The rigid layer 2 can include multiple upper electrodes 4. The soft layer 3 can include multiple lower electrodes 5. The rigid layer 2 can also include multiple inductive coils 6. The multiple upper electrodes 4 and the multiple lower electrodes 5 can form multiple sensing capacitors. The multiple sensing capacitors can be coupled with a single inductive coil 6 or with multiple inductive coils 6 to form multiple resonant circuits. The multiple electrodes of the multiple sensing capacitors can be placed within a certain distance of the center of the rigid layer 2 and the soft layer 3 so that every sensing capacitor can sense deformation of the soft layer at different areas. A reading antenna placed near the sensor can get the resonance frequency of the resonant circuits and measure the surface deformation of the object at different areas.

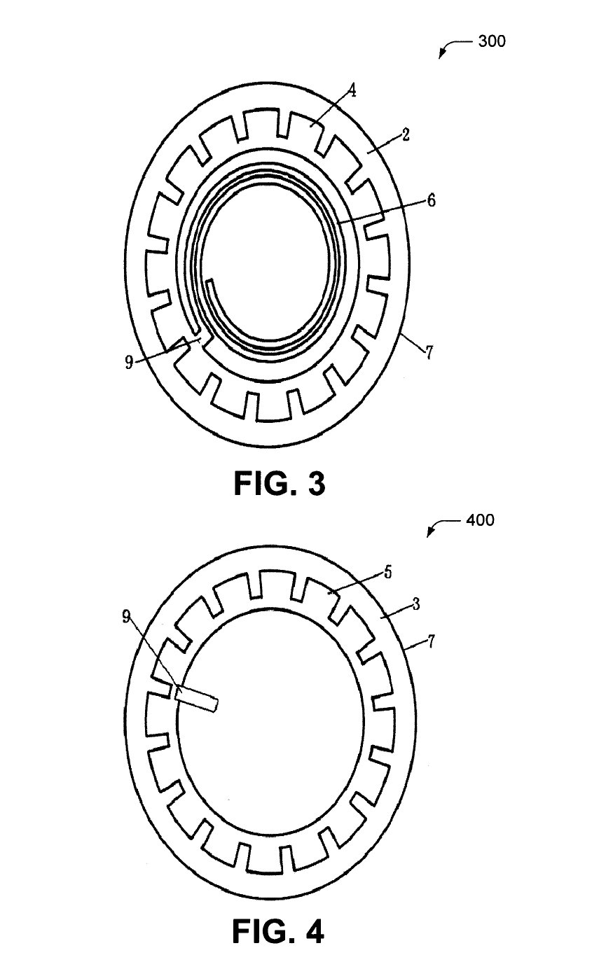

Referring now to FIG. 3, illustrated is a top view (or plan view) 300 of an example of the rigid layer 2 of the surface deformation sensor, according to an embodiment of the disclosure. The rigid layer 2 includes the upper electrode 4 and the inductive coil 6. The rigid layer 2 is bound to the soft layer at the edge 7. The rigid layer 2 has a mechanism 9 that facilitates electrical coupling between the inductive coil 6 and the lower electrode of the soft layer.

The inductive coil 6 can be formed, in an embodiment, by disposing conductive material in a predetermined pattern on in or on the surface of the rigid layer 2. For example, the predetermined pattern can be a spiral pattern. The predetermined pattern can be any pattern that can facilitate creation of a resonant circuit that can resonate proportionally to a change in surface deformation. The inductive coil 6 can be placed closer to the center of the rigid region 2 than the upper electrode 4, in an embodiment. However, the inductive coil 6 need not be placed closer to the center of the rigid region 3 than the upper electrode 4. In an embodiment, the upper electrode 4 can be closer to the center than the inductive coil 6.

Referring now to FIG. 4, illustrated is a top view (or plan view) 400 of an example of the soft layer 3 of the surface deformation sensor, according to an embodiment of the disclosure. The soft layer 3 includes the lower electrode 5. The lower electrode 5 is electrically coupled to the inductive coil of the rigid layer 2 through mechanism 9. The soft layer 3 is bounded to the rigid layer at the edge 7.

Inductive coil 6 need not be fabricated in or on the rigid layer 2. Instead, the inductive coil 6 can be fabricated in or on the soft layer 3. The inductive coil 6, in an embodiment, can be fabricated in or on both the soft layer 3 and the rigid layer 2.

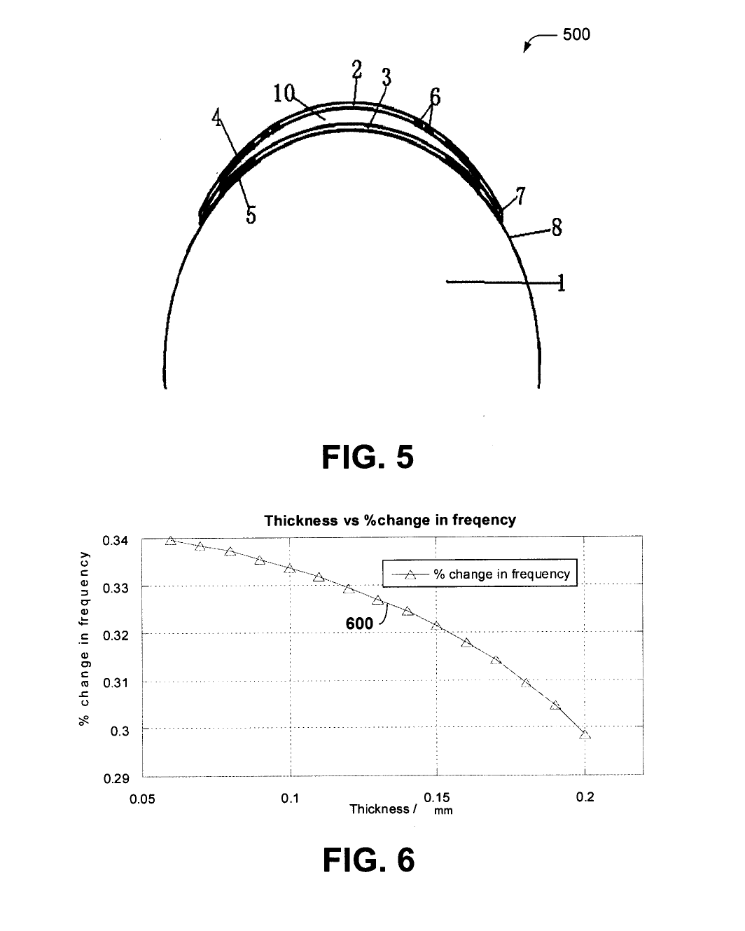

FIG. 5 illustrates an example non-limiting cross sectional view 500 of the surface deformation sensor mounted on the surface of an object, according to an embodiment of the disclosure. The surface deformation sensor as illustrated in FIG. 5 is fabricated as a contact lens that can be applied to the eye 1 with low or no force. In an embodiment, the sensor can be applied to the cornea of the eye 1 to achieve corneal curvature measurement. A soft layer 3 is placed against the eye 1 and deforms in connection with a change in the corneal curvature. The sensor is non-invasive and allows for in vivo measurement of intraocular pressure.

The surface deformation sensor can be used for the diagnosis and treatment of many eye diseases, including glaucoma. The treatment and diagnosis of such eye diseases can be facilitated by knowledge of the intraocular pressure. The intraocular pressure can be determined based on detection of a curvature change in the surface of the cornea. The curvature and corresponding curvature changes can be sensed by changes in the capacitance between a rigid layer 2 with an upper electrode 4 and a soft layer 3 with a lower electrode 5. The upper electrode 4 and the lower electrode 5 can form a resonant circuit with an inductive coil 6 located on the rigid layer 2. The change in capacitance corresponds to a change in resonance frequency. The intraocular pressure can be determined from the corneal curvature with a known relationship between intraocular pressure and corneal curvature based on the resonance frequency. The changes in curvature can be detected by an external reading coil as changes in the resonance frequency.

The sensor of FIG. 5 is a non-invasive method to measure corneal configuration change. The corneal configuration change is correlated to intraocular pressure change. By correlating the configuration change to intraocular pressure change, continuous monitoring of intraocular pressure is possible.

The sensor utilizes a deformable soft layer 3 that can deform conformally with the corneal curvature change. The sensor includes a resonant circuit that produces a resonant frequency that is proportional to the curvature change, which is proportional to the intraocular pressure change. The resonant frequency can be detected wirelessly by a detector and a relationship can be used to determine intraocular pressure. Accordingly, the sensor avoids use of harmful safety hazards, like: elements that require application of force, rigid electrodes, a power element and a silicon amplification and conditioning chip.

The surface deformation of a soft object, such as a cornea, can be tracked by the soft layer. The shape change of the soft layer will change the capacitance and the response to the resonance frequency of the resonant circuit. For example, in an embodiment, the upper electrode surface of the sensor can be about 20 square mm on each side, the dielectric gap cam be about 30 μm, and the inductive coil can have about 3 turns with an inside diameter of about 6 mm and an outside diameter of about 8 mm. With the soft layer undisturbed, the resonant frequency can be on the order of about 123 MHz. According to an example, a 1% increase in capacitance resulting from soft layer deflection will produce a downward shift in resonant frequency to 121.8 MHz. This shift is readily discernible electronically using an external reading antenna and an electric device. From the resonance frequency, the surface deformation of the object can be determined.

To show the relationship between soft layer deformation and layer thickness, a series of sensors with different soft layer thicknesses (from 0.07 mm-0.2 mm or 70 μm to 200 μm) were tested in a porcine eye. FIG. 6 is a graph including a curve 600 illustrating the relationship between deformations of the soft layer with layer thickness, according to an embodiment of the disclosure. Curve 600 of FIG. 6 shows a decreasing signal and corresponding percent change in frequency with a thicker sensor. The decreasing signal is not obvious in the mathematical prediction; however, it can affect the signal significantly.

The decreasing signal with increasing thickness phenomenon may be due to a close contact effect. In other words, a thicker sensor may not contact with the cornea as well as a thinner sensor. The change in the sensor, accordingly, does not totally reflect the change of curvature.

The experimental result showed that the soft layer can deform well with the cornea when the thickness of the soft layer is 100 μm or less. In another embodiment, the soft layer can deform well with the cornea when the thickness of the soft layer is about 70 μm or less. In a further embodiment, the soft layer can deform well with the cornea when the thickness of the soft layer is about 50 μm or less.

The surface deformation sensor fabricated in contact lens form was tested both on bench and in vivo to characterize its electrical, physical, and surgical/biological behaviors. The pressure differences were generated by supplying saline inside the eye chamber. A fiber pressure sensor was used to compare to the surface deformation sensor. An external readout antenna was aligned above the lens on the same axis. The testing configuration was applied to condition the sensors for testing convenience without losing the fidelity of the sensor performance. The surface deformation sensor was tested in porcine eye and rabbit eye. Contact lenses of different sizes were fit to the different eyes according to the average curvature (the porcine eye exhibits an average curvature of 8.9 mm and the rabbit eye has an average curvature of 8.2 mm, so different curvature contact lenses corresponded thereto).

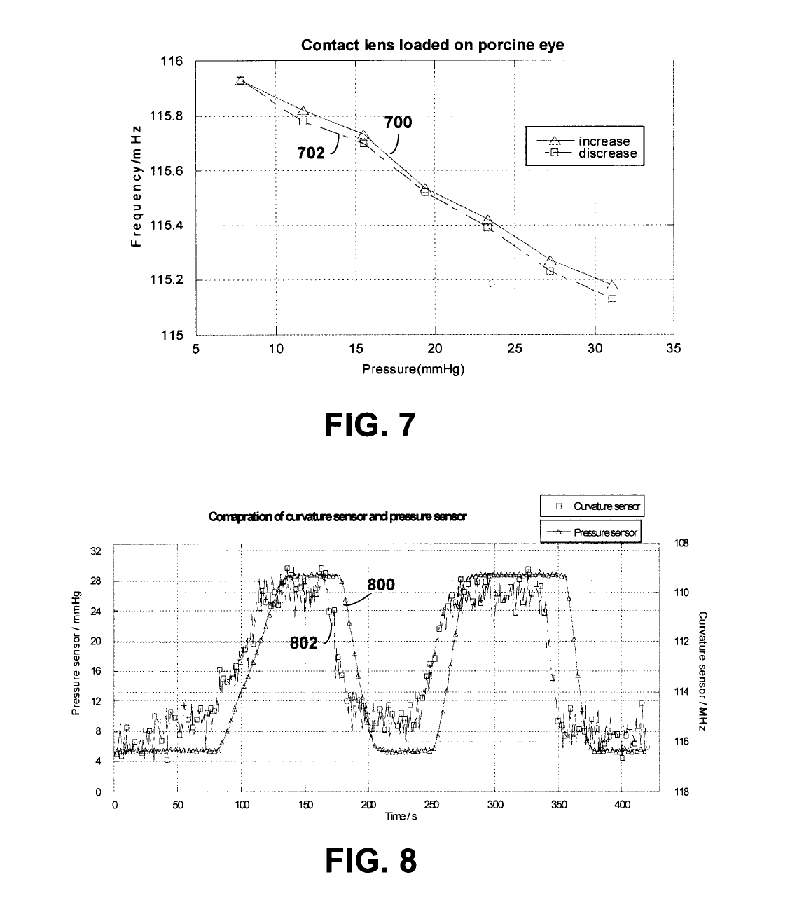

Experimental results successfully verified the feasibility of wireless pressure sensing with the non-invasive contact lens surface deformation sensor. FIG. 7 is a graph with curves 700 and 702 illustrating the relationship between resonance frequency of a resonant circuit of the surface deformation sensor and intraocular pressure, according to an embodiment of the disclosure. The data is root data from the antenna, which took place five times per second with averaging.

In FIG. 7, the triangle line, or curve 700, shows the frequency change with pressure increase and the square line, or curve 702, shows the frequency change with pressure decrease measured with the surface deformation sensor. The sensing is linear and repeatable.

The surface deformation sensor can be utilized to measure intraocular pressure, as shown in FIG. 8. FIG. 8 is a graph with curves 800 and 802 illustrating measurement of intraocular pressure with the surface deformation sensor and measurement of intraocular pressure with a pressure sensor implanted into the eyeball, according to an embodiment of the disclosure. The triangle line, or curve 800, shows data from a pressure sensor implanted into an eye to measure intraocular pressure, while the square line, or curve 802, shows data collected from the surface deformation sensor by measuring cornea curvature change. Comparing the data of the pressure sensor and the surface deformation sensor shows that the surface deformation sensor achieves non-continuous intraocular pressure measurement.

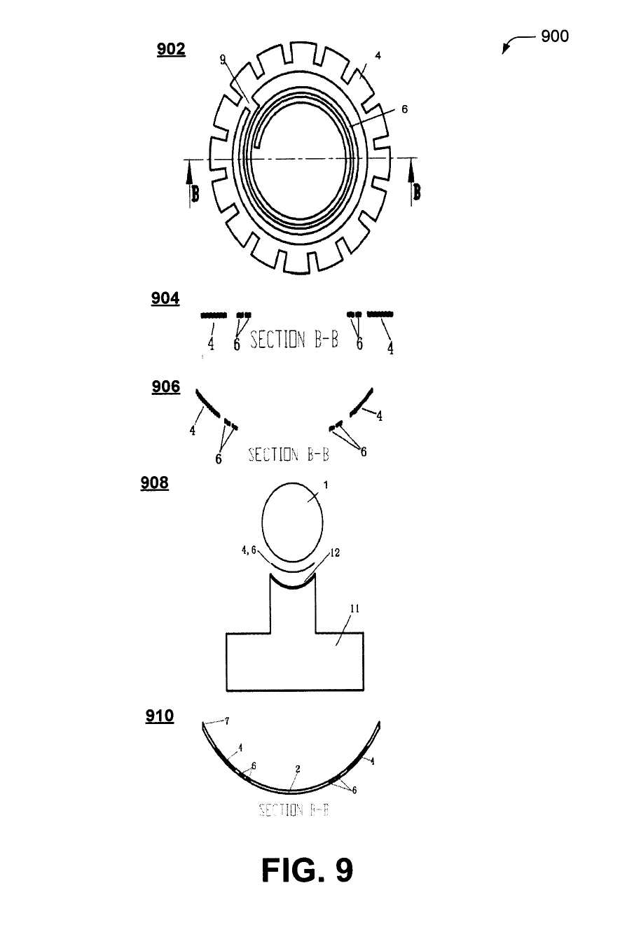

FIGS. 9-11 illustrate methods and/or flow diagrams in accordance with embodiments of this disclosure. For simplicity of explanation, the methods are depicted and described as a series of acts. However, acts in accordance with this disclosure can occur in various orders and/or concurrently, and with other acts not presented and described in this disclosure. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be further appreciated that the methods disclosed in this specification are capable of being stored on an article of manufacture to facilitate transporting and transferring such methods to hardware devices, such as sensors.

Referring now to FIG. 9, illustrated is a process flow diagram of a method 900 for fabricating the rigid layer of the surface deformation sensor, according to an embodiment of the disclosure. The soft layer of the surface deformation sensor can be made in an analogous way.

At 902, a completed rigid layer is shown with the inductive coil 6, the upper electrode 4, and the coupling wire 9. At 904, the inductive coil 6 and the upper electrode 4 are shown from a cross section of line B in 902 as uncurved. At 906, the inductive coil 6 and the upper electrode 6 as shown from a cross section of line B in 902 are curved. At 908, the curvature is confirmed to be substantially similar to the curvature of the eye. At 910, a cross sectional view of the completed rigid layer 2 is shown.

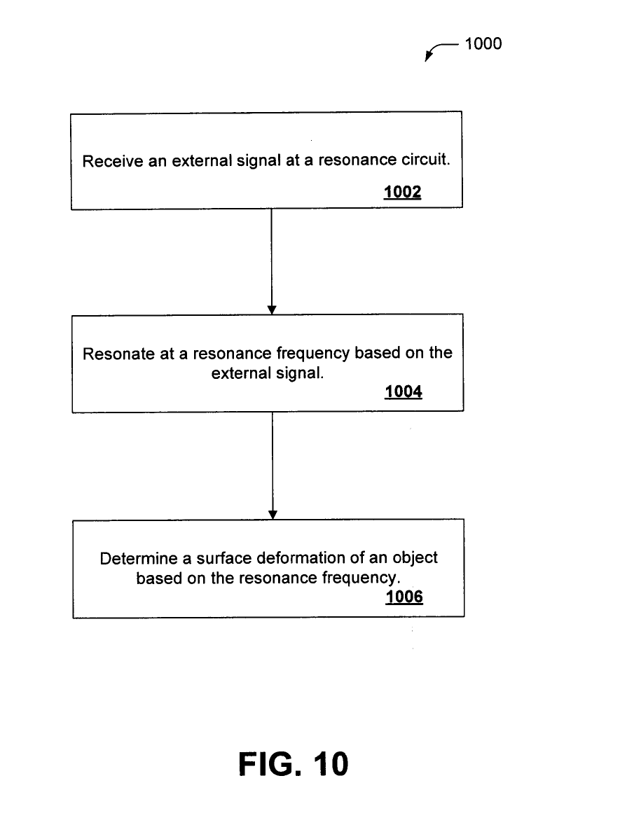

Referring now to FIG. 10, illustrated is a process flow diagram of a method 1000 for determining a surface deformation of an object, according to an embodiment of the disclosure. At 1002, an external signal is received at a resonance circuit. In an embodiment, the external signal is an electromagnetic signal. The resonance circuit includes a sensing capacitor and an inductive coil. The sensing capacitor is made of at least two electrodes: an upper electrode in or on a rigid layer and a lower electrode in or on a soft layer. The rigid layer can include multiple upper electrodes and the soft layer can include multiple lower electrodes to form multiple capacitors. There is a gap between the rigid layer and the soft layer that can be filled with a dielectric material. The inductive coil can be in or on the rigid layer. In an embodiment, the external signal can be received using an external reader and an inductor electromagnet coupled to the inductive coil. The inductive coil can, according to an embodiment, be located on the soft layer. In another embodiment, multiple inductive coils can be coupled to the multiple capacitors to form multiple resonant circuits.

At 1004, the resonance circuit resonates at a resonance frequency based on the external signal. The sensing capacitor can be energized by the external signal and can cause the resonant circuit to resonate at a resonance frequency. For example, the soft layer can be attached to an object. The soft layer can deform conformally with deformations in the object. The deformation of the soft layer can alter the size of the gap, which can alter the capacitance and cause the resonance. At 1006, a surface deformation of an object is determined based on a resonance frequency.

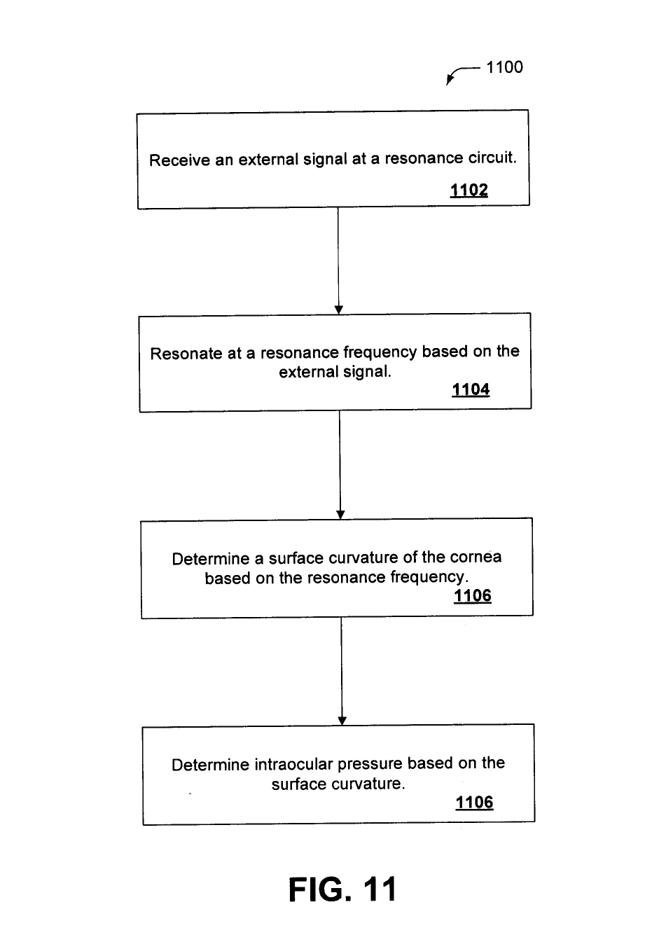

Referring now to FIG. 11, illustrated is a process flow diagram of a method for determining intraocular pressure, according to an embodiment of the disclosure. The sensor can be attached to the eye with minimal pressure. At 1102, an external signal can be received at a resonant circuit. At 1104, the resonant circuit resonates at a resonance frequency based on the external signal. At 1106, a surface curvature of the cornea of the eye is determined based on the resonance frequency. At 1108, the intraocular pressure is determined based on a relationship between the curvature of the cornea and the intraocular pressure. The intraocular pressure can be utilized in the diagnoses and treatment of ocular diseases like glaucoma.

What has been described above includes examples of the embodiments of the subject disclosure. It is, of course, not possible to describe every conceivable combination of components or methods for purposes of describing the claimed subject matter, but it is to be appreciated that many further combinations and permutations of the various embodiments are possible. Accordingly, the claimed subject matter is intended to embrace all such alterations, modifications, and variations that fall within the spirit and scope of the appended claims. Further, the order in which some or all of the process blocks appear in each process should not be deemed limiting. Rather, it should be understood that some of the process blocks can be executed in a variety of orders that are not illustrated in this disclosure. Moreover, the above description of illustrated embodiments of this disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, modules, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. The aforementioned systems, devices, and circuits have been described with respect to interaction between several components and/or blocks. It can be appreciated that such systems, devices, circuits, and components and/or blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, the words “example” or “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In addition, while an aspect may have been disclosed with respect to only one of several embodiments, such feature may be combined with one or more other features of the other embodiments as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A sensor, comprising:
a reference layer comprising an upper electrode and an inductive coil; and
a deformation layer comprising a lower electrode,
wherein:
the lower electrode forms a sensing capacitor with the upper electrode, and
the inductive coil is electrically coupled to the sensing capacitor and produces a resonance in response to an external electromagnetic field.

2. The sensor of claim 1, wherein:
the upper electrode and the lower electrode are conductive thin films, and
the inductive coil is an electrically conductive wire or a semiconductor wire.

3. The sensor of claim 1, wherein:
the upper electrode and the inductive coil are within or on the reference layer, and
the lower electrode is within or on the deformation layer.

4. The sensor of claim 1, wherein:
the reference layer is a rigid film, and
the deformation layer is a soft deformable film.

5. The sensor of claim 1, wherein:
the deformation layer contacts an object, and
the reference layer and the deformation layer have a curvature substantially similar to a curvature of the object.

6. The sensor of claim 1, wherein:
a gap exists between the reference layer and the deformable layer, and
the gap is at least partially filled with a dielectric material.

7. The sensor of claim 6, wherein the dielectric material comprises a gel, a fluid, or a gas.

8. The sensor of claim 6, wherein:
the deformation layer contacts an object and deforms conformally with the object, and
a size of the gap changes with a change in a topology of the object.

9. The sensor of claim 8, wherein the resonance varies with the change in the topology of the object.

10. A bio-compatible sensor, comprising:
a capacitor, comprising:
a rigid layer, comprising an upper electrode;
a soft deformable layer, comprising a lower electrode; and
an inductive coil formed on the rigid layer, electrically coupled to the capacitor to form a resonant circuit that resonates at a resonance frequency that is proportional to the capacitance and is measurable in response to excitation by an electromagnetic signal.

11. The bio-compatible sensor of claim 10, wherein:
the upper electrode and the lower electrode are conductive thin films, and
the inductive coil is an electrically conductive wire or a semiconductor wire.

12. The bio-compatible sensor of claim 10, wherein:
the upper electrode is within or on the rigid layer, and
the lower electrode is within or on the soft deformable layer.

13. The bio-compatible sensor of claim 10, wherein:
the rigid layer is a rigid film; and
the soft deformable layer is a soft deformable film.

14. The bio-compatible sensor of claim 10, wherein:
the soft deformable layer contacts an object, and
the rigid layer and the soft deformable layer have a curvature substantially similar to a curvature of the object.

15. The bio-compatible sensor of claim 14, wherein the object is an eye.

16. The bio-compatible sensor of claim 14,
wherein the soft deformable layer contacts a cornea of an eye, and
a size of the gap changes with a curvature of the cornea.

17. The bio-compatible sensor of claim 10, wherein:
a gap exists between the rigid layer and the soft deformable layer, and
the gap is at least partially filled with a dielectric material.

18. The bio-compatible sensor of claim 16, wherein the dielectric material comprises a bio-compatible gel, a bio-compatible fluid, or a bio-compatible gas.

19. A method, comprising:
receiving an external signal by a resonance circuit, comprising a sensing capacitor and an inductive coil;
energizing the sensing capacitor including resonating the resonance circuit at a resonance frequency based on the external signal;
determining a surface deformation of an object based on the resonating.

20. The method of claim 19, wherein the determining further comprises determining a surface curvature of a cornea of an eye based on the resonating.

21. The method of claim 20, further comprising monitoring a curvature of the cornea based on the surface curvature.

22. The method of claim 20, further comprising determining an intraocular pressure of the eye based on a relationship between the curvature of the cornea and the intraocular pressure.

23. The method of claim 19, wherein the receiving further comprises receiving the external signal using an external reader and an inductor electromagnet coupled with the inductive coil.

24. The method of claim 19, wherein the receiving further comprises receiving the external signal at a plurality of inductors of a plurality of resonance circuits comprising a plurality of sensing capacitors.

25. A system, comprising:
means for energizing a sensing capacitor to resonate a resonance circuit at a resonance frequency based on an external signal; and
means for determining a surface deformation of an object based on resonation at the resonance frequency.

26. The system of claim 25, further comprising means for applying the resonance circuit to the object without damaging the object.

27. The system of claim 25, further comprising means for determining a pressure change inside a hollow object based on the surface deformation.
